(19) United States
(12) Patent Application Publication
(10) Pub. No.: US 2026/0131023 A1
(43) Pub. Date: May 14, 2026

(54) COMPOSITIONS AND METHODS FOR THE PREVENTION AND TREATMENT OF TRANSPLANT REJECTION

(71) Applicant: NxGEN Vector Solutions, LLC, Washington, DC (US)

(72) Inventor: Susan M. Faust, Washington, DC (US)

(21) Appl. No.: 17/807,436

(22) Filed: Jun. 17, 2022

Related U.S. Application Data
(63) Continuation of application No. 16/568,628, filed on Sep. 12, 2019, now abandoned.
(60) Provisional application No. 62/730,735, filed on Sep. 13, 2018.

(51) Int. Cl. A61K 48/00; A61K 35/34; A61K 39/395; A61P 37/06; C07K 16/28; C12N 15/864

(52) U.S. Cl. CPC A61K 48/005; A61K 35/34; A61K 39/39541; A61P 37/06; C07K 16/2812; C12N 15/8645

(57) ABSTRACT

The disclosure relates to compositions and methods useful for gene therapy and for the prevention and treatment of cardiac allograft rejection in the transplant setting.

### CROSS REFERENCE TO RELATED APPLICATIONS

This patent application is a continuation of U.S. patent application Ser. No. 16/568,628, filed on Sep. 12, 2019 and claims priority to U.S. Provisional Ser. No. 62/730,735, filed on Sep. 13, 2018, the entire contents of which is incorporated herein by reference.

### STATEMENT OF RIGHTS TO INVENTIONS MADE UNDER FEDERALLY SPONSORED RESEARCH

Pursuant to 35 U.S.C. § 202(c) it is acknowledged that the U.S. Government has rights in the invention described, which was made with funds from the National Institutes of Health, NIH RO1 HL070613, NIH RO1 AI061469, and T90 DK070071.

### FIELD OF THE INVENTION

This invention relates to the fields of molecular biology and immunology. More specifically, the invention provides compositions and methods useful for cardiac gene therapy and for the prevention and treatment of cardiac allograft rejection in the transplant setting.

### BACKGROUND OF THE INVENTION

Chronic allograft rejection (CR), also referred to as “chronic allograft vasculopathy” or “cardiac transplant vasculopathy”; or “transplant coronary artery disease” or “transplant associated vasculopathy” is the leading cause of late graft failure following organ transplantation. CR is characterized by proliferation of fibroblasts within the graft, which promotes interstitial fibrosis as well as thickening and narrowing of the coronary vessels, a process referred to as transplant associated vasculopathy (TAV). CR is a progressive disease, resulting in deteriorating graft function due to interstitial fibrosis, cardiac hypertrophy and occlusive neointima development. CR may develop in response to multiple mechanisms and evolve from a cell and antibody-mediated phase that leads to allograft tissue damage to a tissue-remodeling phase driven by cytokines and growth factors. In the mouse vascularized allograft model, transient depletion of CD4+ T cells promotes graft survival but leads to CR. Inductive anti-CD4 mAb treatment permits the CD4+ T cells to begin repopulating the periphery 3-4 weeks post-transplantation. As CD 4+ T cells return, donor-reactive T cells are functionally distinct from naïve cells in that these repopulating CD4+ exhibit a transient donor-reactive Th1 and Th2 priming that peaks around day 30 post-transplant. These graft-reactive cells progress to a hyporesponsive state toward the graft, but mount Th2 recall responses and demonstrate accelerated CR if transferred into allograft-bearing SCID mice.

Programmed death-1 (PD-1) is an immune checkpoint receptor on cytotoxic T cells. The PD-1 receptor has two ligands, programmed death ligand-1 (PD-L1) and programmed death ligand-2 (PD-L2). Upregulation of the PD-1 receptor plays a key role in the debilitating process of T-cell exhaustion, as well as being an important factor during the normal immune response to prevent autoimmunity.

### BRIEF SUMMARY OF THE INVENTION

The appended claims define this application. The present disclosure summarizes aspects of the embodiments and should not be used to limit the claims. Other implementations are contemplated in accordance with the techniques described herein, as will be apparent to one having ordinary skill in the art upon examination of the following drawings and detailed description, and these implementations are intended to be within the scope of this application.

In one aspect, the present disclosure includes a method for treating a patient in need of a cardiac transplant. The method comprises introducing a vector into one or more cells contained in a donor tissue, wherein the vector comprises a nucleic acid that encodes a mammalian immunomodulatory protein; and transplanting the donor tissue into the patient. In certain embodiments, the introducing step is performed ex vivo prior to said transplanting. In certain embodiments, the introducing step is performed in situ in the patient subsequent to said transplanting. In certain embodiments, the vector is an adeno-associated viral (AAV) vector, preferably an AAV type 3b or AAV/SASTG vector. In certain embodiments, the vector is a CpG-depleted vector. In certain embodiments, the mammalian immunomodulatory protein is human PD-L1 or a fragment thereof. In certain embodiments, the donor tissue is cardiac tissue. In certain embodiments, the method further comprises administering an anti-CD4 antibody to the patient.

In another aspect, the present disclosure includes a method for improving or promoting a beneficial outcome and/or preventing or reducing the risk of a negative outcome in a transplant patient. The method comprises transplanting a donor tissue into the patient, wherein, prior to said transplanting, (i) donor tissue cells have been transduced with a vector and/or (ii) donor tissue has been incubated in a solution comprising a vector; wherein the vector of (i) and/or (ii) comprises a nucleic acid that encodes a mammalian immunomodulatory protein. In certain embodiments, the beneficial outcome is long-term graft function and/or patient survival. In certain embodiments, the negative outcome is long-term graft failure, chronic rejection due to a chronic alloreactive immune response, transplant coronary artery disease, chronic allograft vasculopathy, occlusive narrowing of coronary vessels, neo-intimal hyperplasia, fibrosis of graft vasculature, and/or microvascular occlusion. In certain embodiments, the solution comprises an organ storage solution. In certain embodiments, the vector is an adeno-associated viral (AAV) vector, preferably an AAV type 3b or AAV/SASTG vector. In certain embodiments, the vector is a CpG-depleted vector. In certain embodiments, the mammalian immunomodulatory protein is human PD-L1 or a fragment thereof. In certain embodiments, the donor tissue is cardiac tissue. In certain embodiments, the method further comprises administering an anti-CD4 antibody to the patient.

In another aspect, the present disclosure includes a donor tissue comprising one or more donor cells transduced with a vector comprising a nucleic acid that encodes a mammalian immunomodulatory protein. In certain embodiments, the vector is an adeno-associated viral (AAV) vector, preferably an AAV type 3b or AAV/SASTG vector. In certain embodiments, the vector is a CpG-depleted vector. In certain embodiments, the mammalian immunomodulatory protein is human PD-L1 or a fragment thereof. In certain embodiments, the donor tissue is cardiac tissue.

In yet another aspect, the present disclosure includes a method for preparing a donor tissue for transplantation into a human host. The method comprises introducing a vector into one or more cells contained in the donor tissue, wherein the vector comprises a nucleic acid that encodes a mammalian immunomodulatory protein. In certain embodiments, the vector is an adeno-associated viral (AAV) vector, preferably an AAV type 3b or AAV/SASTG vector. In certain embodiments, the vector is a CpG-depleted vector. In certain embodiments, the mammalian immunomodulatory protein is human PD-L1 or a fragment thereof. In certain embodiments, the donor tissue is cardiac tissue.

In still another aspect, the present disclosure includes a method for preparing a donor tissue for transplantation into a human host. The method comprises contacting said donor tissue with a composition comprising an organ storage solution and a vector comprising a nucleic acid that encodes a mammalian immunomodulatory protein. In certain embodiments, the vector is an adeno-associated viral (AAV) vector, preferably an AAV type 3b or AAV/SASTG vector. In certain embodiments, the vector is a CpG-depleted vector. In certain embodiments, the mammalian immunomodulatory protein is human PD-L1 or a fragment thereof. In certain embodiments, the donor tissue is cardiac tissue.

In another aspect, the present disclosure includes a method for reducing or inhibiting transplant rejection. The method comprises transplanting a donor tissue prepared according to any method disclosed herein into the host.

In another aspect, the present disclosure includes a composition for transplantation comprising donor tissue and a vector comprising a nucleic acid that encodes a mammalian immunomodulatory protein. In certain embodiments, the vector is an adeno-associated viral (AAV) vector, preferably an AAV type 3b or AAV/SASTG vector. In certain embodiments, the vector is a CpG-depleted vector. In certain embodiments, the mammalian immunomodulatory protein is human PD-L1 or a fragment thereof. In certain embodiments, the donor tissue is cardiac tissue.

In another aspect, the present disclosure includes a composition for preparing donor tissue for transplantation comprising a vector comprising a nucleic acid that encodes a mammalian immunomodulatory protein and an organ storage solution. In certain embodiments, the vector is an adeno-associated viral (AAV) vector, preferably an AAV type 3b or AAV/SASTG vector. In certain embodiments, the vector is a CpG-depleted vector. In certain embodiments, the mammalian immunomodulatory protein is human PD-L1 or a fragment thereof. In certain embodiments, the donor tissue is cardiac tissue.

### BRIEF DESCRIPTION OF THE DRAWINGS

The summary, as well as the following detailed description, is further understood when read in conjunction with the appended drawings. For the purpose of illustrating the disclosed methods and devices, exemplary embodiments of the methods and devices are shown in the drawings, however, the methods and devices are not limited to the specific embodiments disclosed. In the drawings:

FIG. 1 depicts a schematic representation of the acute rejection mouse cardiac allograft experiments, in which allografts were transduced with double-stranded adeno-associated virus vectors that encoded a mouse PD-L1 gene (AAV-PD-L1) or a null vector encoding a scrambled mouse PD-L1 gene sequence and transplanted into fully-MHC disparate recipients.

FIG. 2 depicts an allograft survival curve from the acute rejection mouse cardiac allograft experiments.

FIG. 3 is a bar graph depicting PD-L1 expression at the time of allograft recovery in the mice that received allografts transduced with the AAV PD-L1 vector.

FIGS. 4A and 4B depict histological sections from an exemplary control (null) and experimental animal (PD-L1).

FIG. 5A is a graph depicting flow cytometry analysis of splenocytes from recipient mice. FIG. 5B is a graph depicting ELISPOT analysis of splenocytes from recipient mice.

FIGS. 6A and 6B are bar graphs depicting donor-reactive IgM and IgG production.

FIG. 7A is a graph depicting flow cytometry analysis of graft infiltrating cells. FIG. 7B is a graph depicting differential cell counts of graft infiltrating cells. FIGS. 7C and 7D depict an exemplary ELISPOT from a control (null) and experimental animal (PD-L1).

FIG. 8 is a graph depicting ELISPOT analysis of graft infiltrating cells.

FIG. 9A-9D are bar graphs depicting IFN-gamma, IL-4, IL-17, and FoxP3 expression, respectively.

FIG. 10A-10B depict a schematic representation of the mouse cardiac allograft model, in which prolonged allograft survival can be achieved by transiently depleting recipients of CD4+ cells or by disrupting CD40-CD40L interactions.

FIG. 11 depicts a schematic representation of the CR mouse cardiac allograft experiments, in which allografts were transduced with either double-stranded AAV-PD-L1 or AAV-Null vectors and transplanted into fully-MHC disparate recipients treated with inductive anti-CD4 mAb.

FIG. 12 depicts an allograft survival curve from the CR mouse cardiac allograft experiments.

FIG. 13 is a bar graph depicting PD-L1 expression at the time of allograft recovery in the mice that received allografts transduced with the AAV PD-L1 vector in the CR mouse cardiac allograft experiments.

FIG. 14 is a graph depicting ELISPOT analysis of splenocytes from recipient mice in the CR mouse cardiac model.

FIG. 15 is a graph depicting flow cytometry analysis of splenocytes from recipient mice in the CR mouse cardiac model.

FIGS. 16A and 16B are bar graphs depicting donor-reactive IgM and IgG production in the CR mouse cardiac model.

FIG. 17 is a graph depicting differential cell counts of graft infiltrating cells in the CR mouse cardiac model.

FIG. 18A-18D are bar graphs depicting IFN-gamma, IL-4, IL-17, and FoxP3 expression, respectively, in the CR mouse cardiac model.

FIG. 19A-19C depict morphometric trichrome analysis to assess graft fibrosis. FIG. 19D is a graphical representation of percent fibrosis.

FIG. 20 is a bar graph depicting graft weight in the CR mouse cardiac model.

FIG. 21 is a bar graph depicting cardiac hypertrophy in the CR mouse cardiac model.

### DETAILED DESCRIPTION OF THE INVENTION

While the invention may be embodied in various forms, there are shown in the drawings, and will hereinafter be described, some exemplary and non-limiting embodiments, with the understanding that the present disclosure is to be considered an exemplification of the invention and is not intended to limit the invention to the specific embodiments illustrated. Thus, the following examples are illustrative only and are not a limitation on the present invention.

### I. AAV Vectors for Gene Transfer in the Transplant Setting

In one aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for promoting or improving long-term graft function using such vector or composition.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for promoting allograft recipient survival using such vector or composition.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for preventing or reducing the risk of long-term graft failure using such vector or composition.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for preventing or reducing the risk of chronic rejection, including chronic rejection due to a chronic alloreactive immune response, using such vector or composition.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for preventing or reducing the risk of a heart transplant recipient developing transplant coronary artery disease using such vector or composition.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for preventing, minimizing, or reducing the risk of chronic allograft vasculopathy or cardiac transplant vasculopathy using such vector or composition.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for preventing, minimizing, or reducing the risk of occlusive narrowing of coronary vessels, such as occlusion of intramural and/or epicaridal coronary vessels, neo-intimal hyperplasia, fibrosis of graft vasculature, and/or microvascular occlusion in a heart transplant recipient using such vector or composition.

### II. AAV Vectors for Gene Transfer in the Transplant Setting Administered in the Presence or Absence of Heparin and/or Growth Factors

In one aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for promoting or improving long-term graft function using such vector, wherein the vector or composition is administered to the transplant recipient in the presence or absence of heparin and/or growth factors, such as fibroblast growth factor.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for promoting allograft recipient survival using such vector or composition, wherein the vector or composition is administered to the transplant recipient in the presence or absence of heparin and/or growth factors, such as fibroblast growth factor.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for preventing or reducing the risk of long-term graft failure using such vector or composition, wherein the vector or composition is administered to the transplant recipient in the presence or absence of heparin and/or growth factors, such as fibroblast growth factor.

In another aspect, the present disclosure provides a recombinant viral vector, such as a recombinant adeno-associated viral (AAV) vector and, preferably an AAV type 3b or AAV/SASTG vector, a composition comprising such vector; and/or a method for preventing or reducing the risk of chronic rejection, including chronic rejection due to a chronic alloreactive immune response, using such vector or composition, wherein the vector or composition is administered to the transplant recipient in the presence or absence of heparin and/or growth factors, such as fibroblast growth factor.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for preventing or reducing the risk of a heart transplant recipient developing transplant coronary artery disease using such vector or composition, wherein the vector or composition is administered to the transplant recipient in the presence or absence of heparin and/or growth factors, such as fibroblast growth factor.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for preventing, minimizing, or reducing the risk of chronic allograft vasculopathy or cardiac transplant vasculopathy using such vector or composition, wherein the vector or composition is administered to the transplant recipient in the presence or absence of heparin and/or growth factors, such as fibroblast growth factor.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for preventing, minimizing, or reducing the risk of occlusive narrowing of coronary vessels, such as occlusion of intramural and/or epicaridal coronary vessels, neo-intimal hyperplasia, fibrosis of graft vasculature, and/or microvascular occlusion in a heart transplant recipient using such vector or composition, wherein the vector or composition is administered to the transplant recipient in the presence or absence of heparin and/or growth factors, such as fibroblast growth factor.

In certain embodiments for any aspect disclosed herein, the administration occurs prior to or simultaneous with the transplantation. In certain embodiments for any aspect disclosed herein, the administration occurs after transplantation.

### III. AAV Vectors for Gene Transfer in the Transplant Setting Depleted of Cpg Motifs

In one aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof, a composition comprising such vector; and/or a method for promoting or improving long-term graft function using such vector or composition, wherein the vector and the nucleic acid sequence is modified to reduce the number of CpG motifs.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for promoting allograft recipient survival using such vector or composition, wherein the vector and the nucleic acid sequence is modified to reduce the number of CpG motifs.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for preventing or reducing the risk of long-term graft failure using such vector or composition, wherein the vector and the nucleic acid sequence is modified to reduce the number of CpG motifs.

In another aspect, the present disclosure provides a recombinant viral vector, such as a recombinant adeno-associated viral (AAV) vector and, preferably an AAV type 3b or AAV/SASTG vector, a composition comprising such vector; and/or a method for preventing or reducing the risk of chronic rejection, including chronic rejection due to a chronic alloreactive immune response, using such vector or composition, wherein the vector and the nucleic acid sequence is modified to reduce the number of CpG motifs.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for preventing or reducing the risk of a heart transplant recipient developing transplant coronary artery disease using such vector or composition, wherein the vector and the nucleic acid sequence is modified to reduce the number of CpG motifs.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for preventing, minimizing, or reducing the risk of chronic allograft vasculopathy or cardiac transplant vasculopathy using such vector or composition, wherein the vector and the nucleic acid sequence is modified to reduce the number of CpG motifs.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for preventing, minimizing, or reducing the risk of occlusive narrowing of coronary vessels, such as occlusion of intramural and/or epicaridal coronary vessels, neo-intimal hyperplasia, fibrosis of graft vasculature, and/or microvascular occlusion in a heart transplant recipient using such vector or composition, wherein the vector and the nucleic acid sequence is modified to reduce the number of CpG motifs.

### IV. AAV Vectors for Gene Transfer in the Transplant Setting Depleted of Cpg Motifs and Administered in the Presence or Absence of Heparin and/or Growth Factors

In one aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof, a composition comprising such vector; and/or a method for promoting or improving long-term graft function using such vector or composition, wherein the vector and the nucleic acid sequence is modified to reduce the number of CpG motifs, and wherein the vector or composition is administered to the transplant recipient in the presence or absence of heparin and/or growth factors, such as fibroblast growth factor.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for promoting allograft recipient survival using such vector or composition, wherein the vector and the nucleic acid sequence is modified to reduce the number of CpG motifs, and wherein the vector or composition is administered to the transplant recipient in the presence or absence of heparin and/or growth factors, such as fibroblast growth factor.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for preventing or reducing the risk of long-term graft failure using such vector or composition, wherein the vector and the nucleic acid sequence is modified to reduce the number of CpG motifs, and wherein the vector or composition is administered to the transplant recipient in the presence or absence of heparin and/or growth factors, such as fibroblast growth factor.

In another aspect, the present disclosure provides a recombinant viral vector, such as a recombinant adeno-associated viral (AAV) vector and, preferably an AAV type 3b or AAV/SASTG vector, a composition comprising such vector; and/or a method for preventing or reducing the risk of chronic rejection, including chronic rejection due to a chronic alloreactive immune response, using such vector or composition, wherein the vector and the nucleic acid sequence is modified to reduce the number of CpG motifs, and wherein the vector or composition is administered to the transplant recipient in the presence or absence of heparin and/or growth factors, such as fibroblast growth factor.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for preventing or reducing the risk of a heart transplant recipient developing transplant coronary artery disease using such vector or composition, wherein the vector and the nucleic acid sequence is modified to reduce the number of CpG motifs, and wherein the vector or composition is administered to the transplant recipient in the presence or absence of heparin and/or growth factors, such as fibroblast growth factor.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for preventing, minimizing, or reducing the risk of chronic allograft vasculopathy or cardiac transplant vasculopathy using such vector or composition, wherein the vector and the nucleic acid sequence is modified to reduce the number of CpG motifs, and wherein the vector or composition is administered to the transplant recipient in the presence or absence of heparin and/or growth factors, such as fibroblast growth factor.

In another aspect, the present disclosure provides a recombinant viral vector, such as a recombinant adeno-associated viral (AAV) vector and, preferably an AAV type 3b or AAV/SASTG vector, the vector comprising a nucleic acid sequence encoding a mammalian immunomodulatory protein, preferably human programmed death-ligand 1 (PD-L1) or a fragment thereof; a composition comprising such vector; and/or a method for preventing, minimizing, or reducing the risk of occlusive narrowing of coronary vessels, such as occlusion of intramural and/or epicaridal coronary vessels, neo-intimal hyperplasia, fibrosis of graft vasculature, and/or microvascular occlusion in a heart transplant recipient using such vector or composition, wherein the vector and the nucleic acid sequence is modified to reduce the number of CpG motifs, and wherein the vector or composition is administered to the transplant recipient in the presence or absence of heparin and/or growth factors, such as fibroblast growth factor.

In certain embodiments for any aspect disclosed herein, the administration occurs prior to or simultaneous with the transplantation. In certain embodiments for any aspect disclosed herein, the administration occurs after transplantation.

### V. Heart-in-Box

In certain embodiments, an explanted donor organ (e.g., heart, lung, liver) is maintained in a portable organ perfusion system, such as the OCS™ Heart (TransMedics®), or an ex vivo organ care system such as the OCS (TransMedics®) or an ex vivo organ care system such as the ones described in U.S. Pat. Nos. 8,304,181, 8,465,970, 9,215,867, 9,301,519, 9,457,179, and 9,894,894 the contents of which are herein incorporated by reference in their entirety. In some such embodiments, the organ is transported in the portable organ perfusion system or the ex vivo organ care system. In some such embodiments, the organ is maintained in a physiologic or near physiologic condition in the portable organ perfusion system or the ex vivo organ care system. In some such embodiments, the explanted donor organ is perfused with a recombinant viral vector disclosed herein while being maintained and/or transported in the portable organ perfusion system or the ex vivo organ care system.

### VI. Large Animal Models

In certain embodiments, an explanted donor organ (e.g., heart, lung, liver) is maintained in a portable organ perfusion system, such as the OCS™ Heart (TransMedics®), or an ex vivo organ care system such as the OCS (TransMedics®) or an ex vivo organ care system such as the ones described in U.S. Pat. Nos. 8,304,181, 8,465,970, 9,215,867, 9,301,519, 9,457,179, and 9,894,894 the contents of which are herein incorporated by reference in their entirety. In some embodiments, the explanted donor organ (e.g., heart, lung, liver) is obtained from a large animal such as a pig, a dog, a sheep, a non-human primate or a human. In some such embodiments, the organ is maintained in a physiologic or near physiologic condition in the portable organ perfusion system or the ex vivo organ care system. In some such embodiments, the explanted donor organ is perfused with a recombinant viral vector disclosed herein while being maintained in the ex vivo organ care system.

In certain embodiments, the explanted donor heart from a pig will be arrested, explanted and added to a primed organ care system (OCS, TransMedics Inc., Andover MA) circuit containing washed blood, OCS proprietary solution and perfused with a recombinant viral vector disclosed herein while being maintained in the ex vivo organ care system for a period of time, such as two hours ex vivo perfusion with sampling of the circuit over time. In some embodiments, the explanted donor heart will be re-arrested on the device and the heart will be transplanted heterotopically into a recipient large animal, such as a pig. In some such embodiment, the recipient pig is maintained on immunosuppression for a period of time post-transplant. In some such embodiments, the transplanted heart and the recipient's native heart are explanted and evaluated molecularly for presence, localization, and activity of the protein encoded by the recombinant viral vector disclosed herein throughout the donor heart as well as in the other organs of the recipient. In some such embodiments, the donor heart will be assessed for evidence of acute rejection, chronic rejection, chronic allograft vasculopathy, cardiac transplant vasculopathy, transplant coronary artery disease or allograft acceptance, and systemic donor-reactive innate immune cell, T and B cell responses will be assessed in the recipient animal.

### VII. AAV Vectors for Gene Transfer in the Transplant Setting

In certain embodiments for any aspect or example disclosed herein, a recombinant adeno-associated viral (AAV) vector may be employed. In some such embodiments, the AAV vector is a type 3b vector or derived from a type 3b vector. An exemplary AAV vector based on type 3b is described in Messina, et al., Human Gene Therapy, 23:1031-1042 (October 2012), the entire contents of which is incorporated herein by reference. In some such embodiments, the AAV vector is SASTG, which achieves highly efficient transduction in mouse heart and in cultured neonatal rat cardiomyocytes. SASTG is described in Piacentino, et al., Human Gene Therapy, 23:635-646 (June 2012), the entire contents of which is incorporated herein by reference.

In certain embodiments for any aspect or example disclosed herein, the AAV vector is a modified AAV1, AAV2, AAV4, AAV5, AAV6, AAV7, AAV8, AAV9, AAV10, or AAVrh32.33 vector. In some such embodiments, the modification enhances cardiac transduction. In some such embodiments, the modification enhances transduction in the presence of heparin and/or a growth factor. In some such embodiments, the modification enhances both cardiac transduction and transduction in the presence of heparin and a growth factor. In some such embodiments, the AAV vector harbors a Q263A substitution and/or a T265 insertion relative to the AAV1 sequence or the corresponding alteration(s) in another AAV sequence, such as AAV3b or the like. In some such embodiments, the AAV vector comprises a nucleic acid encoding a Q263A substitution and/or a T265 insertion relative to the AAV1 or the corresponding alteration(s) in another AAV sequence, such as AAV3b or the like.

In certain embodiments for any aspect or example disclosed herein, an AAV vector may be a modified AAV vector having a reduced number of CpG motifs relative to a parent (e.g., a wild type) AAV vector. Exemplary CpG-depleted AAV vectors are disclosed in Faust et al., J Clin Invest, 123(7): 2994-3001 (2013), the entire contents of which is incorporated herein by reference. Exemplary CpG-depleted AAV vectors are also disclosed in US2016/0222414 A1, the entire contents of which is incorporated herein by reference.

It will be readily apparent to those skilled in the art that other suitable modifications and adaptations of the compositions and methods of the invention described herein may be made using suitable equivalents without departing from the scope of the invention or the embodiments disclosed herein.

The compositions and methods described herein will be better understood by reference to the following examples, which are included as an illustration of and not a limitation upon the scope of the invention.

### VIII. EXAMPLE

In this study, adeno-associated virus (AAV) gene transfer of programmed death ligand-1 (PD-L1) into cardiac allografts was employed to assess the impact of intragraft PD-L1 on both acute and chronic rejection of the transplanted organ, systemic donor-reactive T cell response, and allograft acceptance.

### A. PD-L1 Gene Transfer Into Cardiac Allografts in the Acute Rejection Mouse Cardiac Allograft Model

### Overexpression of PD-L1 Does Not Alter Graft Survival in the Acute Rejection Mouse Cardiac Allograft Model

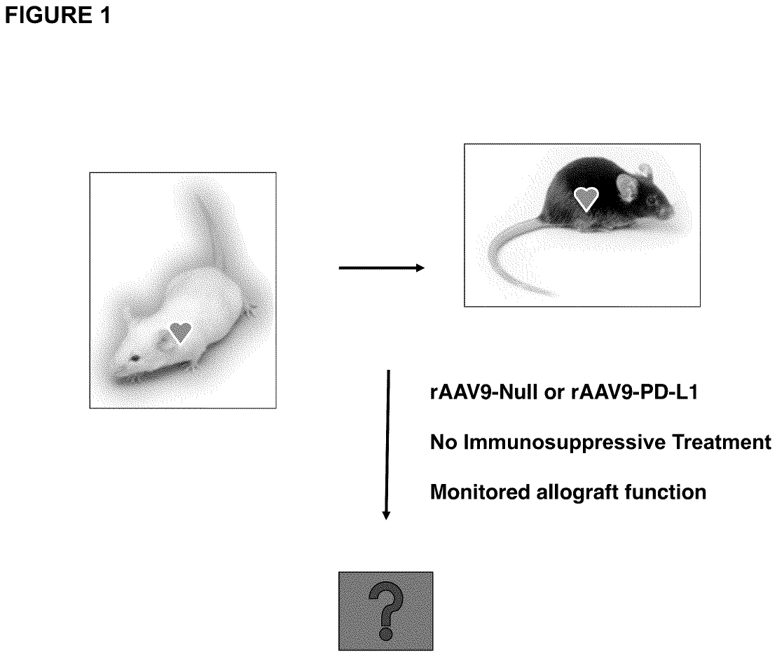

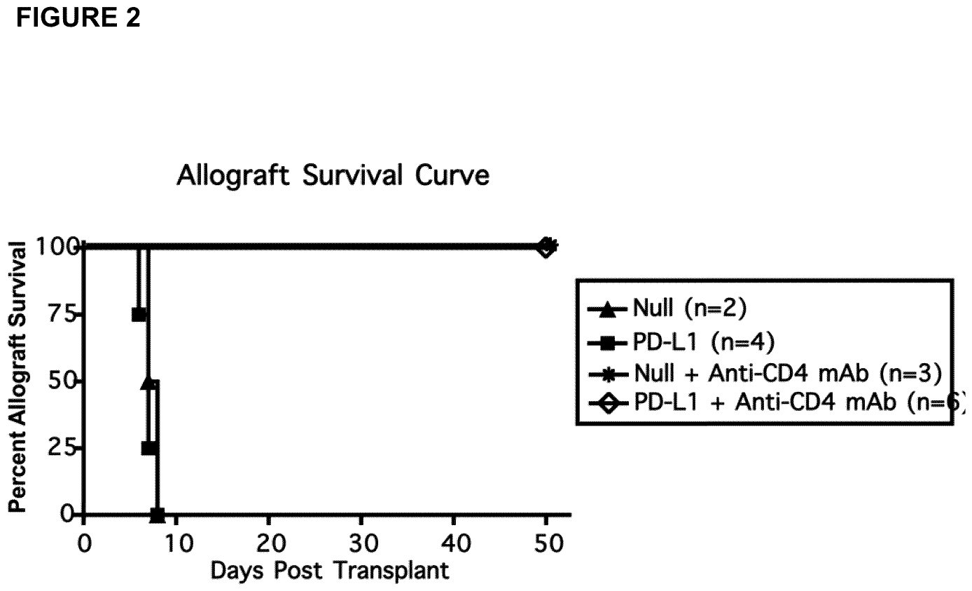

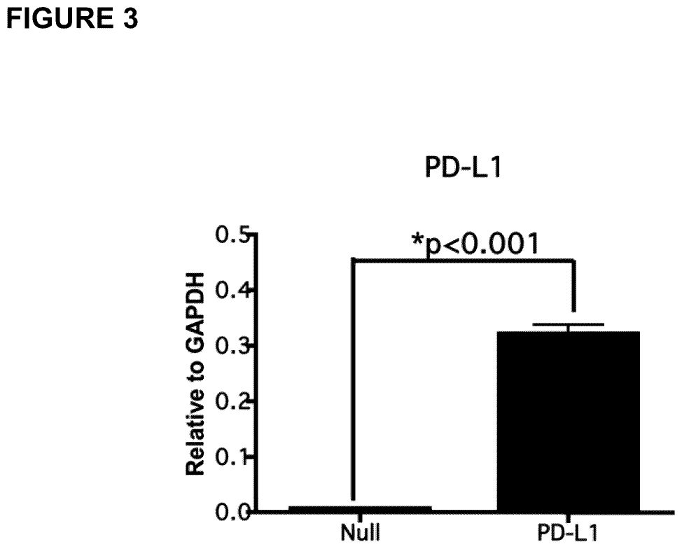

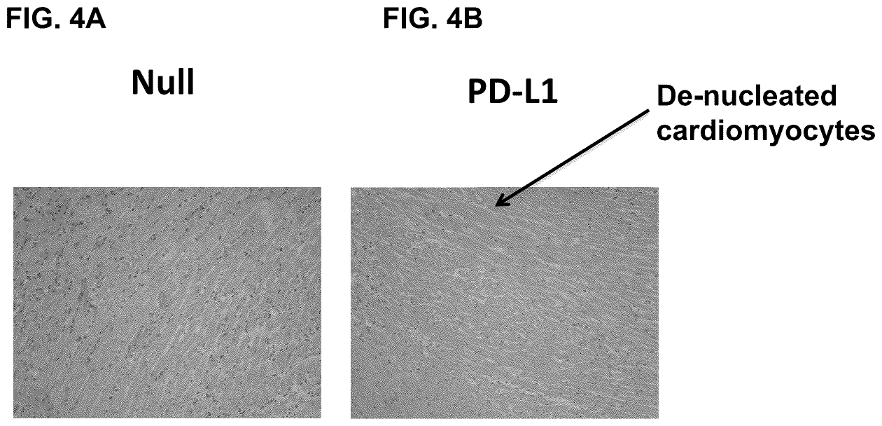

To evaluate efficacy of PD-L1 gene transfer in the acute rejection mouse cardiac allograft model, allografts were transduced with double-stranded adeno-associated virus vectors that encoded a mouse PD-L1 gene (AAV-PD-L1) or a null vector encoding a scrambled mouse PD-L1 gene sequence and transplanted into fully-MHC disparate recipients (FIG. 1). Graft function was monitored by palpation and recipients were recovered at the time of rejection (FIG. 2). All recipients, regardless of whether the mice received an allograft transduced with the null or PD-L1 overexpressing AAV vector, acutely rejected the grafts by day 9 post-transplant, indicating that there was no difference in graft survival between the two groups. Employing mouse PD-L1 specific primers and quantitative RT-PCR, we verified that over-expression of mouse PD-L1 was detected at the time of allograft recovery in the mice that received allografts transduced with the AAV PD-L1 vector (FIG. 3). To investigate the effect of PD-L1 overexpression on graft pathology, cardiac allografts were examined histologically (FIGS. 4A and 4B). Histological examination revealed cellular infiltrate, myocyte necrosis, and hemorrhage—all hallmarks of acute rejection—in both the experimental and control groups indicating that PD-L1 gene transfer did not alter the course or pathological characteristics of acute rejection.

### PD-L1 Gene Transfer Effect on Systemic and Allograft-Localized Inflammatory Responses in the Acute Rejection Mouse Cardiac Model

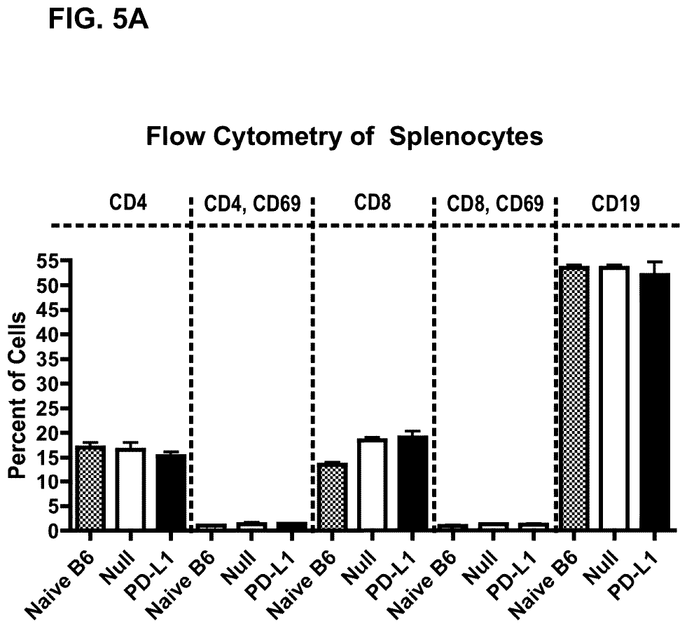

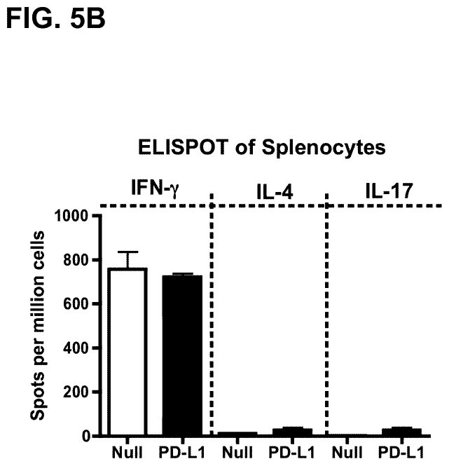

To determine if PD-L1 overexpression within the allografts affected systemic donor-reactive responses, splenocytes from recipient mice were recovered and co-stained with CD4, CD8, CD19, and CD69 antibodies and examined by flow cytometry, or splenocytes were processed for ELISPOT analysis to quantify the number of in vivo primed donor-reactive Th1 (IFN-gamma), Th2 (IL-4), and Th17 responses (FIGS. 5A-5B). Gene transfer of PD-L1 to allografts did not alter the splenocyte cellular composition (FIG. 5A) or abrogate the dominant Th1 associated with acute rejection (FIG. 5B). Th2 and Th17 responses were negligible (FIG. 5B).

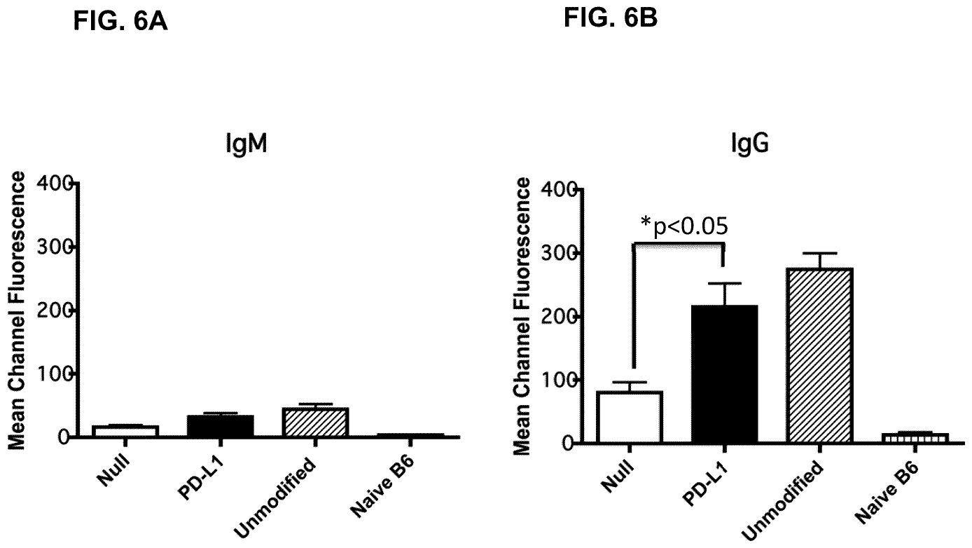

To examine the effect of intragraft PD-L1 overexpression on donor-reactive antibody, we quantified donor-reactive IgM and IgG production by flow cytometry (FIGS. 6A-6B). Recipients of allografts that overexpressed PD-L1 exhibited enhanced donor-reactive IgG production (FIG. 6B).

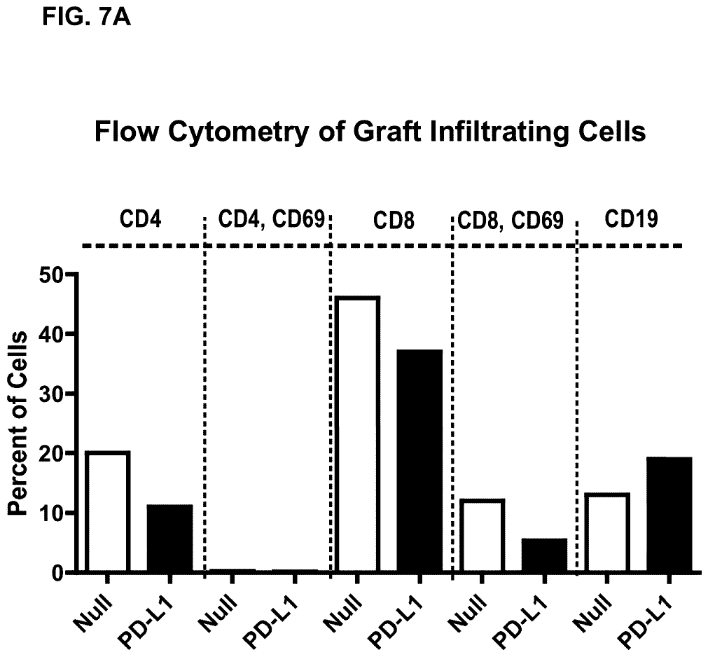

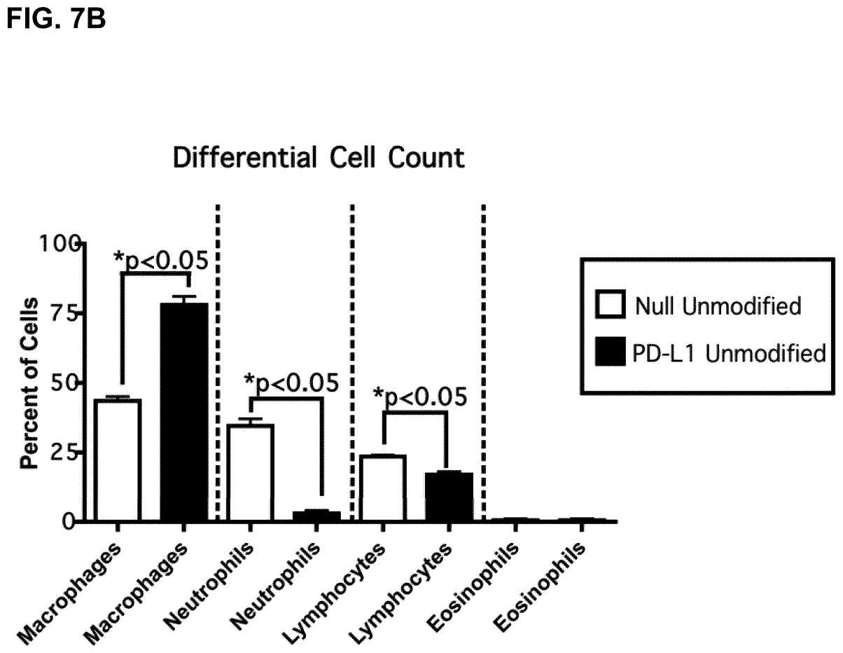

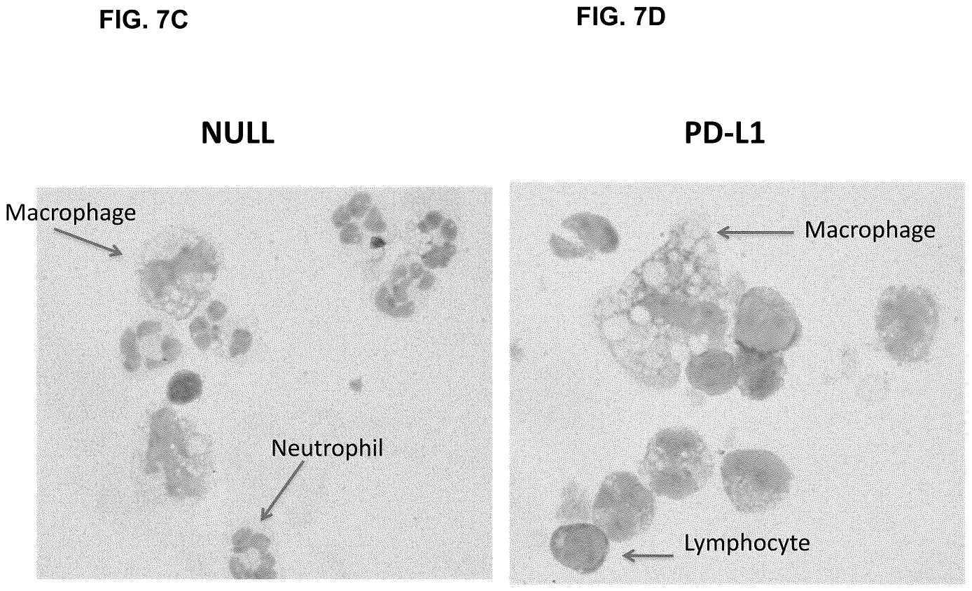

To examine the degree and character of cellular infiltrate within the allografts of the mice that received the null or PD-L1 overexpressing AAV vector, intragraft immune cells were recovered and processed for a differential cell count employing cytospin analysis, co-stained with CD4, CD8, CD19, and CD69 antibodies and examined by flow cytometry, or processed for ELISPOT analysis (FIGS. 7A-7D). PD-L1 gene transfer resulted in a trend toward a reduced percentage of graft-infiltrating activated CD4+ and CD8+ T cells (FIG. 7A). Differential cell counts of graft infiltrating cells confirmed the reduced lymphocyte population, and also revealed a reduced neutrophil population (FIGS. 7B-7D). While the lymphocyte and neutrophil populations were significantly reduced in the allografts of recipients that received the AAV-PD-L1 vector, an enhanced macrophage population was observed compared to control recipients (FIG. 7B).

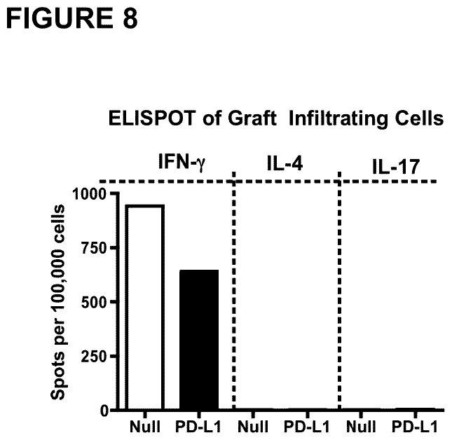

To determine if PD-L1 gene transfer within the allografts affected localized donor-reactive immune responses, ELISPOT was employed to quantify the number of in vivo primed donor-reactive Th1 (IFN-gamma), Th2 (IL-4), and Th17 (IL-17) responses of graft infiltrating cells. PD-L1 overexpression resulted in a trend toward a reduction in Th1 responses (FIG. 8). Th2 and Th17 responses were negligible (FIG. 8)

### Effect of Intragraft PD-L1 Gene Transfer on Gene Expression

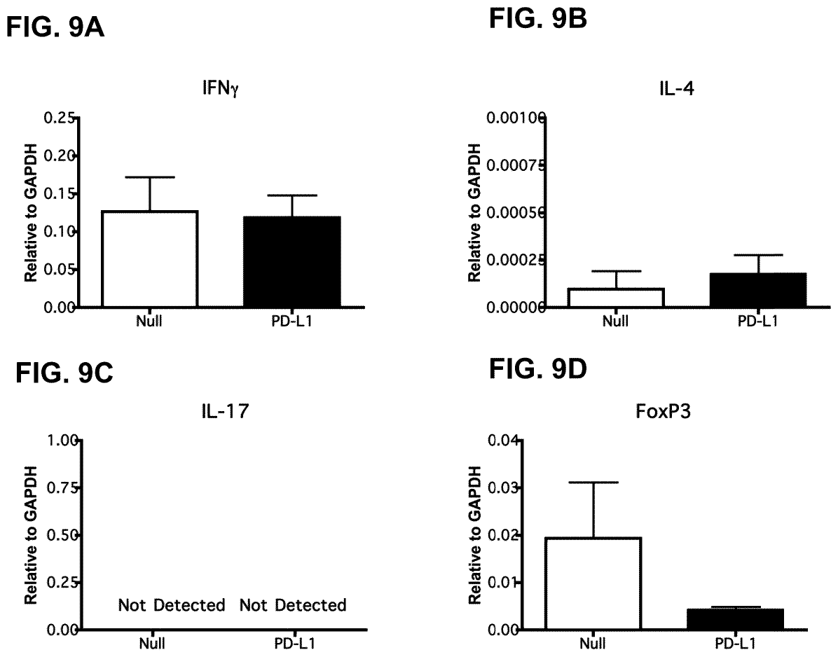

To assay for the effect of PD-L1 gene transfer on genes such as IFN-gamma, IL-4, IL-17, and FoxP3, RNA was isolated from the allografts and quantitative RT-PCR was performed (FIGS. 9A-9D). In recipients transduced with AAV-PD-L1, IFN-gamma, IL-4 and FoxP3 levels were comparable between AAV-Null and AAV-PD-L1 transduced allografts (FIGS. 9A, 9B and 9D), while IL-17 transcript expression was negligible in both groups (FIG. 9C).

### B. PD-L1 Gene Transfer Into Cardiac Allografts in the Chronic Rejection Mouse Cardiac Allograft Model

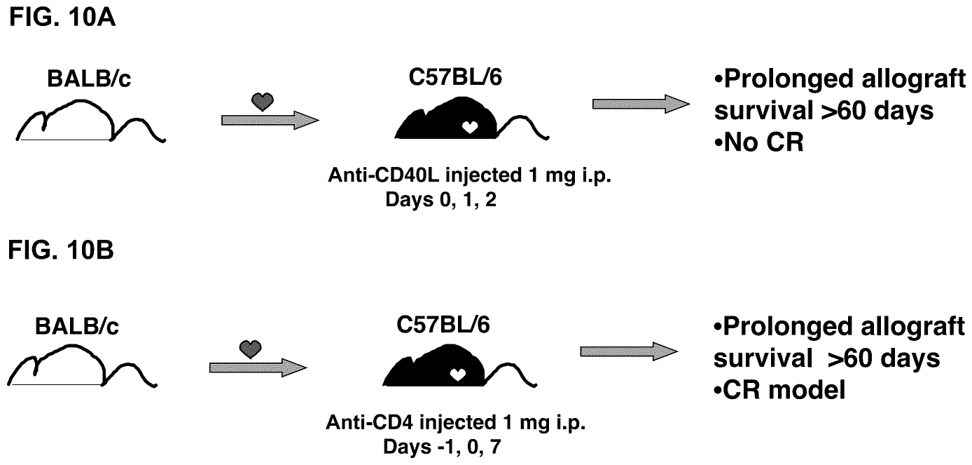

In the mouse cardiac allograft model, prolonged allograft survival can be achieved by transiently depleting recipients of CD4+ cells or by disrupting CD40-CD40L interactions. However, these two inductive therapies differ with respect to the development of CR. Allografts in mice treated with anti-CD40L mAb remain free of CR, while grafts in mice transiently depleted of CD4+ cells develop CR (FIGS. 10A and 10B). While graft-reactive T cells remain in a hyporesponsive state in both settings, recall responses of cells from these groups differ in that dominant Th2 responses are mounted by mice that are depleted of CD4+ cells, while Th1 responses are mounted by recipients given anti-CD40L mAb therapy (Cobbold SP, Castejon R, Adams E, Zelenika D, Graca L, Humm S, Waldmann H. Induction of FoxP3+ regulatory T cells in the periphery of T cell receptor transgenic mice tolerized to transplants. J. Immunol. 2004; 172:6003-6010.)

### PD-L1 Gene Transfer Into Cardiac Allografts Improves Long-Term Graft Function

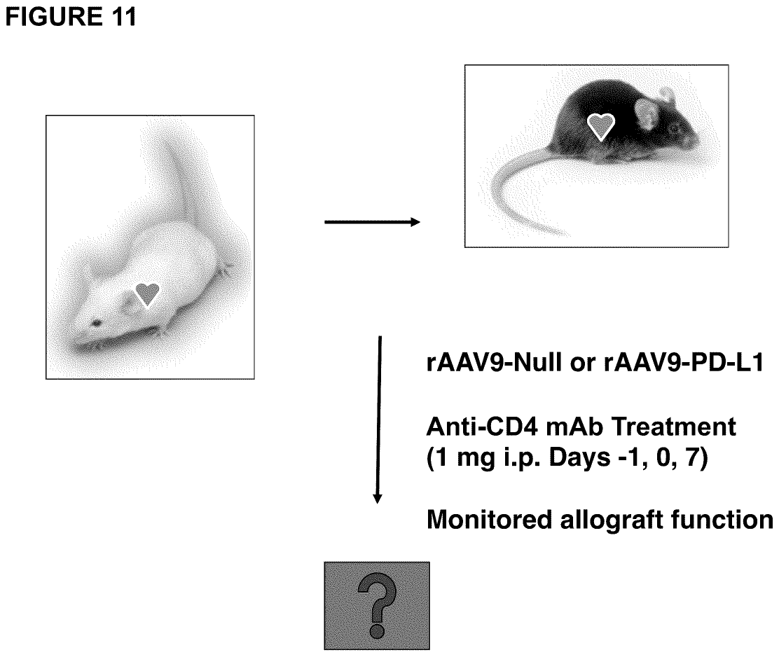

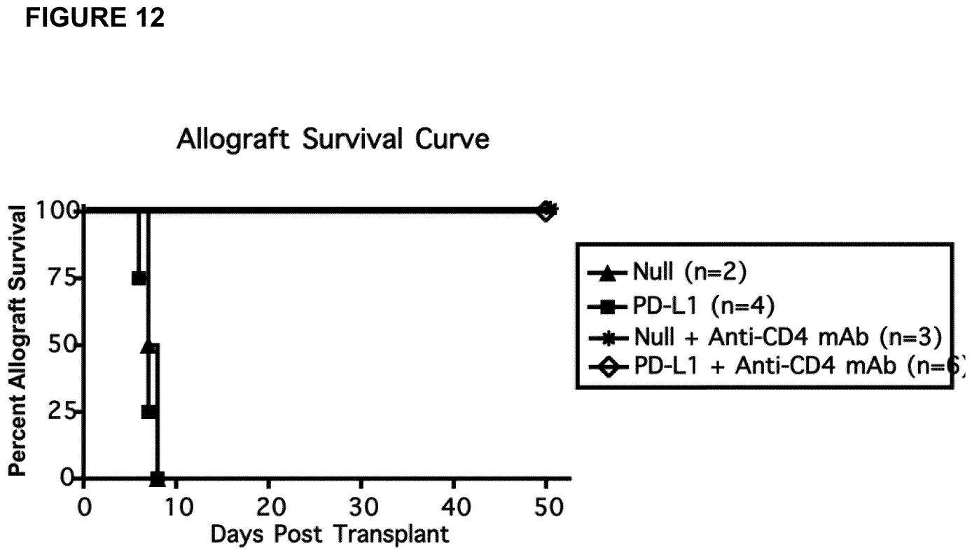

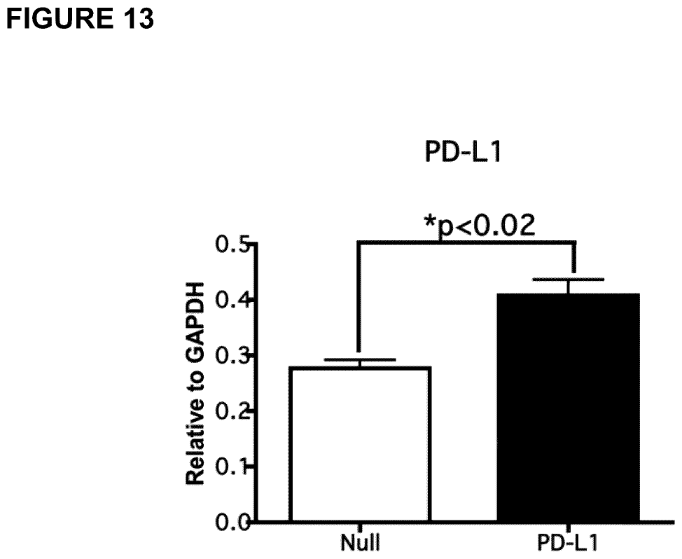

To evaluate the efficacy of PD-L1 gene transfer in the CR mouse allograft model, allografts were transduced with either double-stranded AAV-PD-L1 or AAV-Null vectors and transplanted into fully-MHC disparate recipients treated with inductive anti-CD4 mAb (FIG. 11). No difference in graft survival was observed; all allografts were functioning when recovered on day 50 post-transplant at the completion of the experiment (FIG. 12). However, the recipient mice of AAV-PD-L1 transduced allografts demonstrated improved cardiac function as monitored by palpation. Employing mouse PD-L1 specific primers and quantitative RT-PCR, we verified that over-expression of mouse PD-L1 was detected within the cardiac allografts (FIG. 13). Long-term PD-L1 expression was also detected in the AAV-Null transduced allografts at day 50 post-transplant, albeit at a significantly reduced level compared to the allografts that were transduced with the AAV-PD-L1 vector. These results demonstrate the efficacy and tissue localization of PD-L1 gene transfer into allografts using the AAV-PD-L1 vector and the persistence of transgene expression as well as a native mechanism whereby the transplanted allograft upregulates PD-L1 to defend against the recipient immune system.

### PD-L1 Gene Transfer Effect on Systemic and Allograft-Localized Inflammatory Responses in the CR Mouse Cardiac Allograft Model

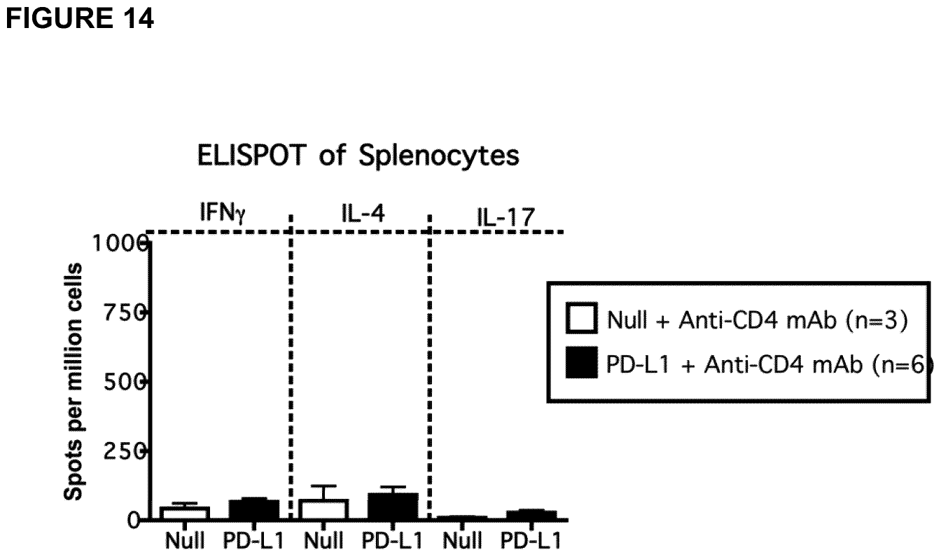

As previously reported, donor-reactive Th1 and Th2 responses are suppressed in recipients transiently depleted of CD4+ T cells. To determine if PD-L1 overexpression within the allografts affected systemic donor-reactive responses in the CR mouse cardiac model, splenocytes from recipient mice were recovered and processed for ELISPOT analysis to quantify the number of in vivo primed donor-reactive Th1 (IFN-gamma), Th2 (IL-4), and Th17 responses (FIG. 14). Consistent with these findings, inductive anti-CD4 mAb therapy resulted in hyporesponsiveness in both groups. These data indicate that overexpression of PD-L1 does not alter the systemic hyporesponsiveness toward the allograft in recipients initially depleted of CD4+ T cells normally observed in the CR mouse cardiac allograft model.

### Flow Cytometry, B Cells and Antibody Production:

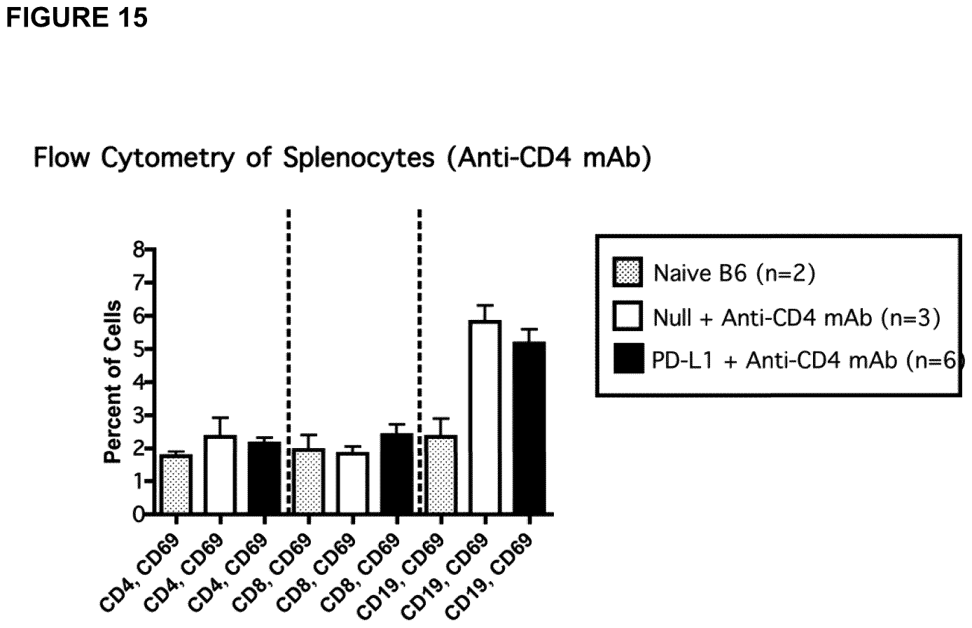

To determine if PD-L1 overexpression within the allografts affected systemic donor-reactive responses in the CR mouse cardiac model, splenocytes from recipient mice were recovered and co-stained with CD4, CD8, CD19, and CD69 antibodies and examined by flow cytometry (FIG. 15). Gene transfer of PD-L1 to allografts did not alter the splenocyte cellular composition compared to the control group (FIG. 15), although both groups did exhibit enhanced activated B cell populations compared to a naïve control.

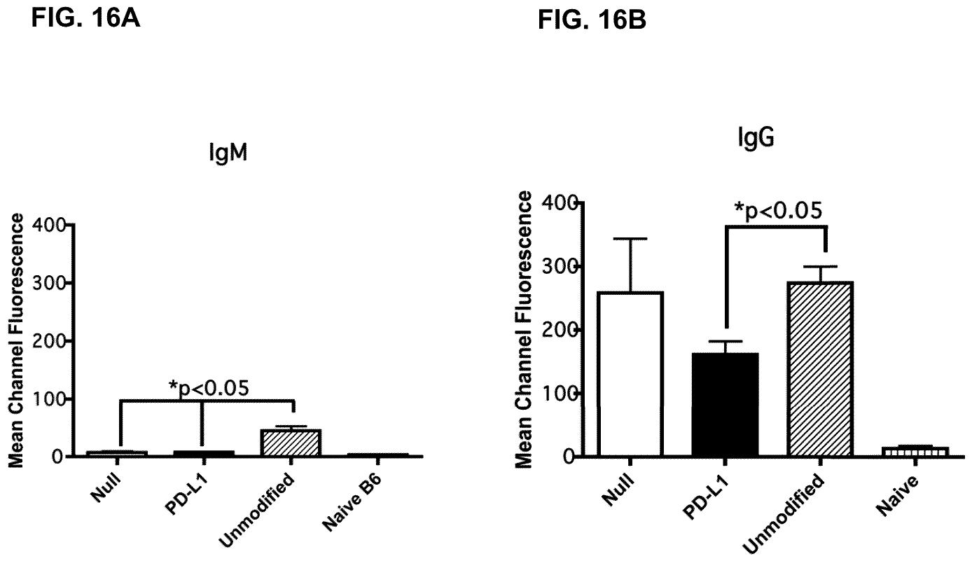

To examine the effect of intragraft PD-L1 overexpression on donor-reactive antibody, we quantified donor-reactive IgM and IgG production by flow cytometry (FIGS. 16A and 16B). Recipients of allografts that overexpressed PD-L1 exhibited minimal IgM donor-reactive responses and similar donor-reactive IgG production. Donor-reactive IgG production in both control and experimental groups were enhanced compared to a naïve control

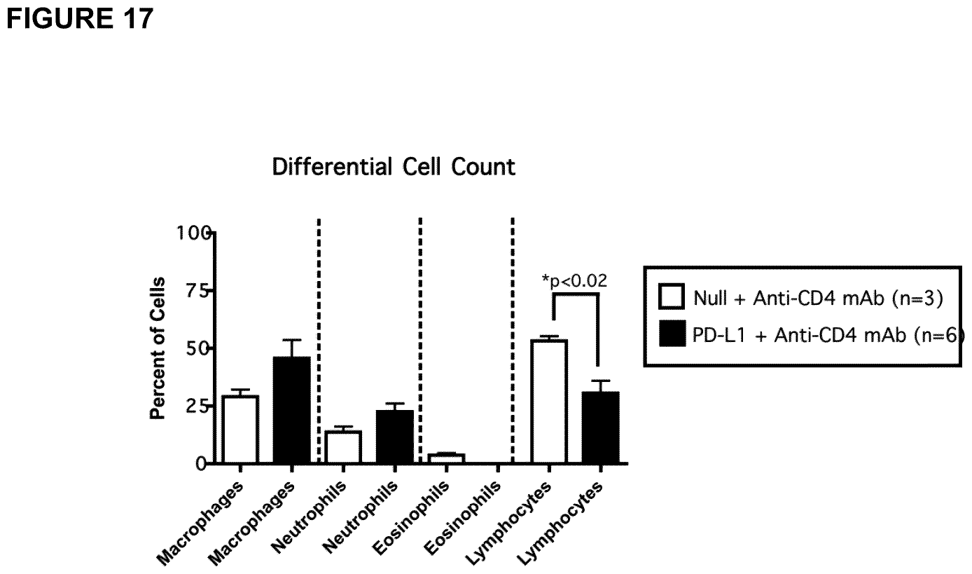

To examine the degree and character of cellular infiltrate within the allografts of the mice that received the null or PD-L1 overexpressing AAV vector, intragraft immune cells were recovered and processed for a differential cell count employing cytospin analysis (FIG. 17). PD-L1 gene transfer resulted in a reduced percentage of graft-infiltrating lymphocytes compared to control (FIG. 17).

### Effect of Intragraft PD-L1 Gene Transfer on Gene Expression

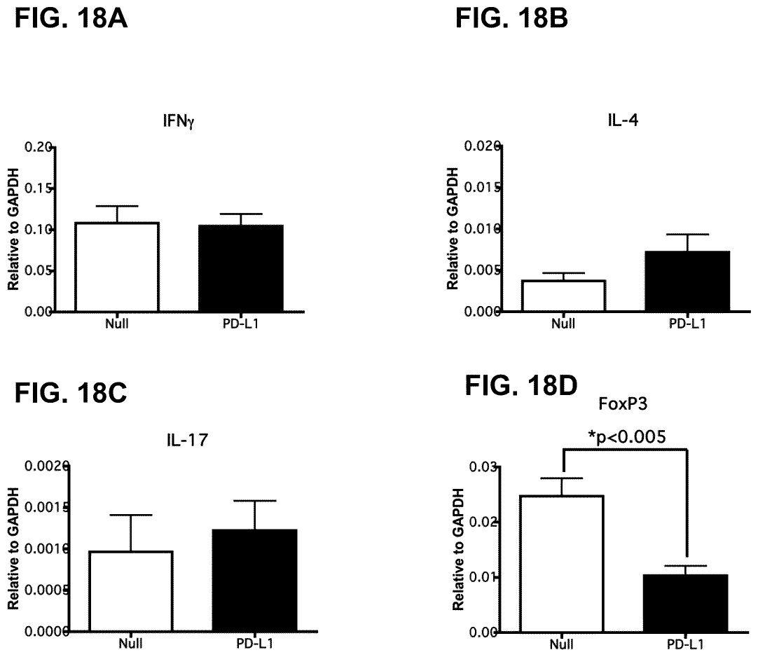

To assay for the effect of PD-L1 gene transfer on genes such as IFN-gamma, IL-4, IL-17, and FoxP3, RNA was isolated from the allografts and quantitative RT-PCR was performed (FIGS. 18A-18D). In recipients transduced with AAV-PD-L1, FoxP3 (p<0.005) transcript levels were significantly reduced compared to control allografts (FIG. 18D). In contrast, IFN-gamma, IL-4, IL-17 levels were comparable between AAV-Null and AAV-PD-L1 transduced allografts (FIGS. 18A-18C).

### Overexpression of PD-L1 Significantly Attenuates Graft Fibrosis and Hypertrophy

While short-term survival rates of solid organ transplants continue to improve, long-term graft survival remains constant. In vascularized organ allografts, the primary cause for late graft failure is CR. CR is characterized by proliferation of fibroblasts within the graft, which promotes interstitial fibrosis as well as thickening and narrowing of the coronary vessels, a process referred to as transplant associated vasculopathy. CR may develop in response to multiple mechanisms and evolve from a cell and antibody-mediated phase that leads to allograft tissue damage to a tissue-remodeling phase driven by cytokines and growth factors.

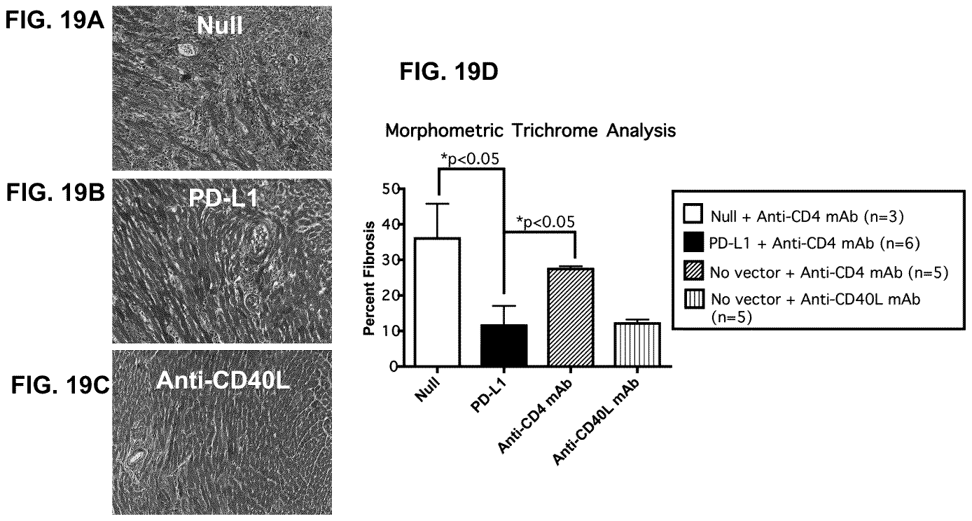

To investigate the effect of PD-L1 gene transfer on graft fibrosis, quantitative morphometric trichrome analysis was performed (FIGS. 19A-19D). Assessment of allograft fibrosis revealed that intragraft PD-L1 overexpression resulted in a significant reduction of collagen deposition compared to Null vector transduced grafts (p<0.05) (FIGS. 19A, 19B, and 19D). In the mouse cardiac allograft model, prolonged allograft survival can be achieved by disrupting CD40-CD40L interactions. Allografts in mice treated with anti-CD40L mAb remain free of CR (FIGS. 19B, 19C, and 19D). Allografts from recipients that received the AAV-PD-L1 vector exhibited comparable collagen deposition to anti-CD40L mAb treated recipients whose grafts remain free of CR (FIG. 19D).

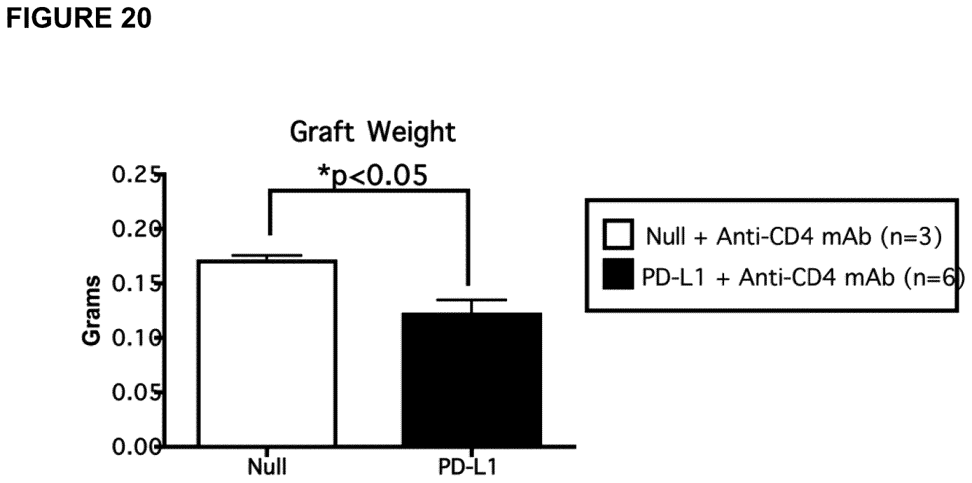

A significant reduction in graft weight in recipients of allografts transduced with rAAV-PD-L1 was also observed and consistent with reduced collagen deposition as measured by morphometric trichrome analysis (FIG. 20). These data indicate that localized PD-L1 overexpression can significantly attenuate CR.

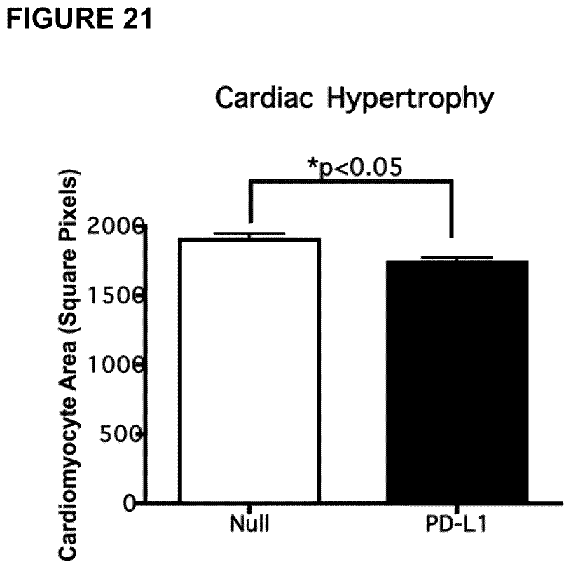

Cardiac hypertrophy is defined as an increase in the heart mass (Marian AJ, Genetic determinants of cardiac hypertrophy. Curr Opin Cardiol 2008; 23:199-205). An increase in the size of the cardiac myocytes, as opposed to the number, is the primary basis of cardiac hypertrophy. To evaluate the effect of intragraft PD-L1 gene transfer on cardiac hypertrophy, cardiomyocyte size was measured employing histologic analysis (Diaz, JA, Booth, AJ, Lu, G, Wood SC, Pinsky DJ, Bishop DK. Critical Role for IL-6 in hypertrophy and fibrosis in chronic cardiac allograft rejection. Am J Transplant 2009; 9:1773-1783). Reduced cardiac hypertrophy was observed in Ad-PD-L1 transduced grafts (FIG. 21). These findings indicate that intragraft PD-L1 overexpression can attenuate both fibrosis and hypertrophy in CR allografts.

In summary, these data show that combined transient immunosuppression and targeting of PD-1 through AAV-mediated PD-L1 gene transfer can modulate pathologic donor-reactive T and B cell responses in vivo and block chronic allograft rejection, effectively attenuating graft fibrosis to a level comparable to a mouse model of allograft transplantation whose grafts remain free of CR.

Thus, AAV gene transfer of PD-L1 modulates the immune response towards the transplanted organ in acute and chronic cardiac allograft rejection.

In this application, the use of the disjunctive is intended to include the conjunctive. The use of definite or indefinite articles is not intended to indicate cardinality. In particular, a reference to “the” object or “a” and “an” object is intended to denote also one of a possible plurality of such objects. Further, the conjunction “or” may be used to convey features that are simultaneously present instead of mutually exclusive alternatives. In other words, the conjunction “or” should be understood to include “and/or”. The terms “includes,” “including,” and “include” are inclusive and have the same scope as “comprises,” “comprising,” and “comprise” respectively.

While certain of the preferred embodiments of the present invention have been described and specifically exemplified above, it is not intended that the invention be limited so such embodiments. Various modifications may be made thereto without departing from the scope of the present invention.

1. A method for treating a patient in need of a cardiac transplant, the method comprising:
preparing donor cardiac tissue according to the method of claim 16; and
transplanting the donor tissue into the patient.

2. The method of claim 1, wherein said introducing is performed ex vivo prior to said transplanting.

3. The method of claim 1, wherein said introducing is performed in situ in the patient subsequent to said transplanting.

4. The method of claim 1, wherein the vector is an adeno-associated viral (AAV) vector, preferably an AAV type 3b or AAV/SASTG vector.

5. The method of claim 1, wherein the vector is a CpG-depleted vector.

6. The method of claim 1, wherein the mammalian immunomodulatory protein is human PD-L1 or a fragment thereof.

7. A method for improving or promoting a beneficial outcome and/or preventing or reducing the risk of a negative outcome in a transplant patient, the method comprising:
transplanting a donor tissue into the patient, wherein, prior to said transplanting, (i) donor tissue cells have been transduced with a vector and/or (ii) donor tissue has been incubated in a solution comprising a vector; wherein the vector of (i) and/or (ii) comprises a nucleic acid that encodes a mammalian immunomodulatory protein.

8. The method of claim 7, wherein the beneficial outcome is long-term graft function and/or patient survival.

9. The method of claim 7, wherein the negative outcome is long-term graft failure, chronic rejection due to a chronic alloreactive immune response, transplant coronary artery disease, chronic allograft vasculopathy, occlusive narrowing of coronary vessels, neo-intimal hyperplasia, fibrosis of graft vasculature, and/or microvascular occlusion.

10. The method of claim 7, wherein the solution comprises an organ storage solution.

11. The method of claim 7, wherein the vector is an adeno-associated viral (AAV) vector, preferably an AAV type 3b or AAV/SASTG vector.

12. The method of claim 7, wherein the vector is a CpG-depleted vector.

13. The method of claim 7, wherein the mammalian immunomodulatory protein is human PD-L1 or a fragment thereof.

14. The method of claim 7, wherein the donor tissue is cardiac tissue.

15. The method of claim 7, wherein further comprising administering an anti-CD4 antibody to the patient.

16. A composition for preparing donor tissue for transplantation comprising a recombinant associated viral (AAV) vector comprising a nucleic acid that encodes human PD-L1 or a fragment thereof and an organ preservation solution comprising one or more preservatives.

17. The composition of claim 16, wherein the vector is an SASTG AAV vector.

18. The composition of claim 16, wherein the vector is a CpG-depleted vector.

19. (canceled)

20. The composition of claim 16, wherein the donor tissue is cardiac tissue.

21. The composition of claim 17, wherein the AAV vector is a AAV type 3b vector.
